SEQUENCE LISTING (6 sequences)

SEQ ID NO: 1 - DNA, 449 bp, Raphanus sp.
  source 1..449: mol_type = other DNA
ctcaacccggagttccacctgaagaagcaggggctgcggtagctgctgaatcttctactggtacatggacaactgtgtggaccgatgggcttaccagccttgaccgttacaaaggccgatgctaccacatcgagcccgttccaggagaagaaactcaatttattgcgtatgtagcttaccccttagacctttttgaagaagggtctgttactaacatgtttacctcaattgtgggtaacgtatttgggttcaaagccctggctgctctacgtctagaggatctgcgaatccctccggcttatactaaaactttccagggaccacctcatggtatccaagttgaaagagataaattgaacaagtatggacgtcccctattaggatgtactattaaacctaagttggggttatccgcgaagaactatggtagagcagtttatgaatgtcta

SEQ ID NO: 2 - DNA, 451 bp, Raphanus sp.
  source 1..451: mol_type = other DNA
ctcaacccggagttccacctgaagaagcaggggctgcggtagctgctgaatcttctactggtacatggacaaaactgtgtggaccgatgggcttaccagccttgatcgttacaaaggacgatgctaccacatcgagcccgttccaggagaagaaaatcaatttattgcttatgtagcttaccctttagacctttttgaagaaggttcggttactaacatgtttacctcgattgtgggtaatgtatttgggttcaaagccctggctgctctacgtctagaggatctgcgaatccctcctgcttatactcaaactttccagggaccacctcatggtatccaagttgaaagagagaaattgaacaagtatggacgtcccctattaggatgtactattaaacctaaattggggttatccgcgaagaactgtggtagagcagtttatgaatgtcta

SEQ ID NO: 3 - DNA, 50 bp, synthetic construct
  source 1..50: mol_type = other DNA
tcgtcggcagcgtcagatgtgtataagagacagcctacgggnggcwgcag

SEQ ID NO: 4 - DNA, 55 bp, synthetic construct
  source 1..55: mol_type = other DNA
gtctcgtgggctcggagatgtgtataagagacaggactachvgggtatctaatcc

SEQ ID NO: 5 - DNA, 53 bp, synthetic construct
  source 1..53: mol_type = other DNA
tcgtcggcagcgtcagatgtgtataagagacagatgcgatacttggtgtgaat

SEQ ID NO: 6 - DNA, 54 bp, synthetic construct
  source 1..54: mol_type = other DNA
gtctcgtgggctcggagatgtgtataagagacagtcctccgcttattgatatgc

(19) United States
(12) Patent Application Publication
(10) Pub. No.: US 2026/0248706 A1
(43) Pub. Date: Aug. 27, 2026

(54) COSMETIC COMPOSITION COMPRISING DNA EXTRACTED FROM RADISH SPROUTS

(71) Applicant: HYUNDAI BIOLAND CO., LTD., Cheongju-si, (KR)

(72) Inventors: Ji Yeon LEE, Cheongju-si (KR); Jua KIM, Cheongju-si (KR); Byung Kuk KIM, Cheongju-si (KR); So Yoon CHA, Sejong-si (KR); Hye Min KANG, Seoul (KR)

(73) Assignee: HYUNDAI BIOLAND CO., LTD., Cheongju-si, (KR)

(21) Appl. No.: 19/550,880

(22) Filed: Feb. 26, 2026

(30) Foreign Application Priority Data
Feb. 27, 2025 (KR) 10-2025-0025798
Dec. 3, 2025 (KR) 10-2025-0189443

(51) Int. Cl. A61K 8/60; A61Q 19/00

(52) U.S. Cl. CPC A61K 8/606; A61Q 19/00

(57) ABSTRACT

Proposed is radish sprout-derived PDRN that strengthens a skin basement membrane and barrier, protects the skin from reactive oxygen species (ROS), and enhances skin defense by further activating the NRF-2 pathway under oxidative stress. Accordingly, the composition containing the radish sprout-derived PDRN can be usefully used to maintain a healthy and firm skin environment, and can be widely applied as a cosmetic for antioxidant and skin barrier protection purposes.

### CROSS-REFERENCE TO RELATED APPLICATION

This application claims priority to and the benefit of Korean Patent Application Nos. 10-2025-0025798, filed on Feb. 27, 2025 and 10-2025-0189443, filed on Dec. 3, 2025, the disclosures of which are incorporated herein by reference in their entireties.

### INCORPORATION BY REFERENCE OF SEQUENCE LISTING

The content of the electronically submitted sequence listing, file name: Q318020_sequence listing as filed; size: 7,501 bytes; and date of creation: Feb. 26, 2025, filed herewith, is incorporated herein by reference in its entirety.

### BACKGROUND

### 1. Field

The present disclosure relates to a cosmetic composition containing DNA extracted from radish sprouts for skin antioxidant efficacy and skin barrier strengthening.

### 2. Discussion of Related Art

DNA contains genetic information and is a substance that activates adenosine receptors in cell membranes to produce effects such as skin regeneration, wound healing, and anti-inflammatory effect. Recently, polydeoxyribonucleotide (PDRN), which is prepared from DNA extracted from salmon sperm, has been actively used in the pharmaceutical and cosmetic fields for the purpose of skin regeneration and tissue repair. PDRN has been confirmed to have effects such as promoting growth factor secretion, improving angiogenesis and blood flow, and having anti-inflammatory action through A2 receptor stimulation, and is utilize in various treatments such as diabetic foot ulcers, scars, vascular insufficiency, and hair loss. However, PDRN is mainly prepared from salmon semen, an animal-based raw material, making it unsuitable for vegan products and cruelty-free cosmetics, and there are limitations such as the use of organic solvents in the existing extraction process. Accordingly, research is underway to extract DNA from natural plant resources such as seaweed, but plants and seaweed contain many impurities such as chlorophyll, and there is a risk that the DNA will lose its function as it is reduced to a low molecular weight during the extraction process. Therefore, it is important to develop a technology to extract and utilize DNA with minimal damage and fewer impurities from non-animal raw materials.

Meanwhile, skin cells are exposed to external stimuli such as ultraviolet rays and reactive oxygen species (ROS), causing waste products to accumulate, which leads to a decline in cell function and a weakening of the skin barrier. To activate these skin cells, the development of cosmetic compositions that activate various physiological functions by linking non-animal DNA is still insufficient. Therefore, there is a need to develop a technology that can stably secure functional DNA from new raw materials and utilize the above mechanisms in a complex manner.

### RELATED ART DOCUMENT

### Patent Document

(Patent Document 1) Korean Patent No. 10-2237543 (Title of the Invention: Method of preparing DNA derived from plant of the genus Aloe, and anti-aging and anti-inflammatory composition comprising DNA derived from plant of the genus Aloe as active ingredient, Applicant: Moachem Co., Ltd., Registration Date: Apr. 1, 2021).

### SUMMARY

The purpose of the present disclosure is to provide a cosmetic composition containing DNA extracted from radish sprouts for skin antioxidant efficacy and skin barrier strengthening.

The present disclosure may relate to a cosmetic composition for skin antioxidation containing DNA extracted from radish sprouts.

The PDRN has a reactive oxygen species scavenging ability and can enhance the activation of a nuclear factor erythroid 2-related factor 2 (NRF-2) induced by oxidative stress.

In addition, the PDRN has a skin barrier strengthening efficacy and can enhance protein expression of laminin-5 or occludin or enhance gene expression of flaggrin.

Hereinafter, the present disclosure will be described in more detail.

In the present disclosure, the radish sprouts may be sprouts grown by germinating radish seeds (seeds of radish, seeds of daikon). That is, radish sprouts and radish microgreens are in a microgreen stage before 1-2 main leaves emerge from the radish seeds. At this time, the radish seeds can be used when the new shoots have grown for 1 to 7 days, preferably 3 to 7 days, and the length of the radish shoots is not greatly limited. A cultivation method for the radish sprouts is not limited; any method such as hydroponic cultivation or soil cultivation may be employed. The radish sprouts are distinguished from radish greens, which are mature radish leaves.

The radish sprout PDRN of the present disclosure at a concentration of 50 to 250 μg/ml increases the expression of laminin-5 protein by 2.7 to 4.0 times. In addition, occludin (OCDN) protein expression is increased by 1.6 to 4.2 times at 25 to 100 μg/ml of the radish sprout PDRN. At this time, even with only 25 μg/ml of the radish sprout PDRN, the expression of occludin (OCDN) is restored to a normal state before cell damage. At the same time, filaggrin (FLG) gene expression also recovers from oxidative stress-induced cell damage to a state similar to that of the normal cell group due to the radish sprout PDRN at concentrations of 25 to 100 μg/mL. At this time, the filaggrin gene expression increases by approximately 1.4 to 1.7 times compared to an oxidative stress-induced group.

The scavenging ability of ROS generated by the oxidative stress may be 25 to 27% at 250 μg/ml of radish sprout PDRN. In particular, the radish sprout PDRN of the present disclosure at 50 to 250 μg/ml can enhance the activation of NRF-2 induced by the oxidative stress by 1.3 to 1.5 times. At this time, the oxidative stress causes NRF-2 activation, and the PDRN further enhances NRF-2 activation and moves it into the nucleus, thereby upregulating the expression of various antioxidant factors.

Accordingly, the present disclosure a pharmaceutical composition containing the radish sprout PDRN and a pharmaceutical excipient for skin antioxidant efficacy and skin barrier strengthening. The PDRN may be added to the pharmaceutical composition of the present disclosure in an amount of 0.001 to 100 wt %, preferably 0.001 to 30 wt %.

In addition, the pharmaceutical composition may be formulated and used in the form of oral formulations such as powders, granules, tablets, capsules, suspensions, emulsions, syrups, aerosols, and the like, external preparations, suppositories, and sterile injection solutions according to existing methods. Carriers, excipients and diluents that may be included in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. In the case of formulation, it is prepared using diluents or excipients such as fillers, extenders, binders, wetting agents, disintegrating agents and surfactants usually used. A solid preparation for oral administration include tablets, pills, powders, granules, capsules, and the like, and such a solid preparation may be prepared by mixing the extract of the present disclosure with at least one excipients, for example, starch, calcium carbonate, sucrose or lactose, gelatin, or the like. In addition to a simple excipient, lubricant such as magnesium stearate and talc are also used. A liquid preparation for oral administration may include suspensions, liquids for internal use, emulsions, and syrups, and in addition to water and liquid paraffin, which are commonly used simple diluents, various excipients such as wetting agents, sweeteners, aromatics, and preservatives. The preparation for parenteral administration includes sterilized aqueous solutions, non-aqueous solutions, suspensions, emulsions, lyophilized preparations, and suppositories. As the nonaqueous solvent and suspending agent, propylene glycol, polyethylene glycol, vegetable oils such as olive oil, injectable esters such as ethyl oleate, and the like may be used. As the suppository base, witepsol, Macrogol, Tween 61, cacao butter, laurin butter, glycerogeratin, and the like may be used.

A dosage of the pharmaceutical composition of the present disclosure will vary depending on the age, sex, and body weight of a subject to be treated, the specific disease or pathological condition to be treated, the severity of the disease or pathological condition, the route of administration, and the judgment of the prescriber. The determination of the dosage based on these factors is within the level of those skilled in the art, and the dosage is generally in the range of 0.01 mg/kg/day to approximately 2000 mg/kg/day. A more preferred dosage is 1 mg/kg/day to 500 mg/kg/day. The administration may be performed once a day, or divided into several doses per day. The above dosages do not limit the scope of the disclosure in any respect.

The pharmaceutical composition of the present disclosure may be administered to mammals such as rats, livestock, and humans through various routes. All routes of administration may be envisaged, for example, oral, rectal, intravenous, intramuscular, subcutaneous, intrauterine, epidural, or intracerebral injection. The composition of the present disclosure has almost no toxicity or side effects, so it is a drug that may be safely used even for long-term use for preventive purposes.

A formulation of the cosmetic composition of the present disclosure may be any form commonly prepared in the art, including essence, lotion, cream, emulsion, pack, hand cream, foot cream, body lotion, lip balm, lipstick, eye shadow, eyeliner, eyebrow pencil, blusher, highlighter, general toner, skin, cream, serum, cosmetic soap, softening toner, medicated toner, body cleanser, cleansing foam, cleansing lotion, gel, cleansing oil, cleansing cream, cleansing tissue, cleansing water, mask pack, and other hair products; in particular, various hair cosmetic products for hair growth, hair loss improvement, scalp improvement, and the like; more specifically, hair soap, hair shampoo, hair rinse, hair serum, hair treatment, hair essence, hair water, hair tonic, hair lotion, hair emulsion, hair cream, hair massage cream, hair wax, hair pack, hair oil, hair drying agent, hair preservative, hair dye, hair bleach, hair gel, hair glaze, hair mousse, hair spray, hair ampoule, and the like.

More specifically, when a formulation of the cosmetic composition of the present disclosure is a paste, cream or gel, animal fiber, vegetable fiber, wax, paraffin, starch, tragacanth, a cellulose derivative, polyethylene glycol, silicone, bentonite, silica, talc, or zinc oxide can be used as a carrier component. When the formulation of the cosmetic composition of the present disclosure is a powder or spray, lactose, talc, silica, aluminum hydroxide, calcium silicate or polyamide powder may be used as a carrier component, and in particular, in the case of a spray, a propellant such as chlorofluorohydrocarbon, propane-butane, or dimethyl ether may be additionally included. When the formulation of the cosmetic composition of the present disclosure is a solution or emulsion, a solvent, solvating agent or emulsifying agent is used as a carrier component, and may be water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, or fatty acid esters of sorbitan. When the formulation of the cosmetic composition of the present disclosure is a suspension, a liquid diluent such as water, ethanol or propylene glycol, an ethoxylated isostearyl alcohol, a suspending agent such as polyoxyethylene sorbitol ester, microcrystalline cellulose, aluminum metahydroxide, bentonite, agar, tragacanth, or the like may be used as a carrier component. When the formulation of the cosmetic composition of the present disclosure is a surfactant-containing cleanser, aliphatic alcohol sulfates, aliphatic alcohol ether sulfates, sulfosuccinic acid monoester, acethionate, imidazolinium derivatives, methyl taurate, sarcosinate, fatty acid amide ether sulfates, alkylamidobetaine, fatty alcohols, fatty acid glyceride, fatty acid diethanolamide, vegetable oil, lanolin derivatives, or ethoxylated glycerol fatty acid esters may be used as a carrier component. The cosmetic composition of the present disclosure may further include excipients including fluorescent substances, fungicides, hydrotropes, moisturizers, fragrances, fragrance carriers, proteins, solubilizers, sugar derivatives, sunscreens, vitamins, plant extracts, and the like. The components may be selected in an amount that does not impair the inherent effects of the cosmetic composition depending on the formulation or purpose of use. The amount of the components added may be, for example, 0.1 to 10 wt %, preferably 0.1 to 6 wt %, based on the total weight of the composition, but is not limited thereto.

### BRIEF DESCRIPTION OF THE DRAWINGS

The above and other objects, features, and advantages of the present disclosure will become more apparent to those of ordinary skill in the art by describing exemplary embodiments thereof in detail with reference to the accompanying drawings, in which:

FIG. 1 shows the electrophoresis results of the radish sprout-derived PDRN of the present disclosure.

FIGS. 2A to 2C show the results of confirming the cytotoxicity of the radish sprout PDRN of the present disclosure.

FIG. 3 shows the results confirming the efficacy of the radish sprout PDRN of the present disclosure in enhancing laminin-5 protein expression.

FIG. 4A shows the results of fluorescent immunostaining imaging and numerical analysis confirming the efficacy of the radish sprout PDRN of the present disclosure in enhancing occludin (OCDN) protein expression, and FIG. 4B shows the results confirming the efficacy of the radish sprout PDRN of the present disclosure in enhancing filaggrin (FLG) gene expression.

FIG. 5 shows the results of confirming the ROS scavenging ability of the radish sprout PDRN of the present disclosure.

FIG. 6 shows the results confirming the efficacy of the radish sprout PDRN of the present disclosure in enhancing NRF-2 protein expression.

### DETAILED DESCRIPTION OF EXEMPLARY EMBODIMENTS

Hereinafter, preferred embodiments of the present disclosure will be described in more detail. However, the present disclosure is not limited to the embodiments described herein, and may be embodied in other forms. Rather, the present disclosure is provided to ensure that the content introduced herein is thorough and complete, and to fully convey the concept of the disclosure to those skilled in the art.

### Example 1. Preparation of Radish Sprout-Derived PDRN

To obtain the radish sprout PDRN, the radish sprouts were first washed and then the raw radish sprouts were finely pulverized.

The raw radish sprouts were mixed with a buffer solution containing 500 mM tris(hydroxymethyl)aminomethane hydrochloride, 100 mM potassium phosphate, 170 mM ethylenediaminetetraacetic acid, 0.1 M sodium chloride, 0.1 M sodium acetate, and 100 mM sodium hydroxide, and containing 10 mM ascorbic acid and 0.5 wt % sodium dodecyl sulfate. At this time, purified water for preparing the buffer solution was added at 40 times the weight of the raw radish sprouts. After that, the mixture was heated at 60° C. with stirring for 1 hour.

Once the raw radish sprout tissue and DNA were separated, impurities other than DNA, such as the tissue, were removed. Additionally, impurities were primarily removed by centrifuging at 4° C. and 7500 rpm for 10 minutes to eliminate the precipitated solids. In addition, after most of the impurities were removed, 99.9% ethanol was added to the remaining solution to precipitate the radish sprout-derived DNA in the solution, and the ethanol concentration in the solution was adjusted to 50 (v/v) %. Again, centrifugation was performed again to discard the ethanol and obtain only the precipitate containing DNA. The obtained precipitate was dissolved in purified water and re-precipitated with ethanol (purity of 99.99% or higher). This was centrifuged, and the precipitate alone was rehydrated in purified water. This process was repeated 2 to 4 times to remove ethanol components from the precipitate, purifying only the DNA components to obtain high pure radish sprout PDRN.

### Example 2. Gene Sequence Analysis of Radish Sprout-Derived PDRN

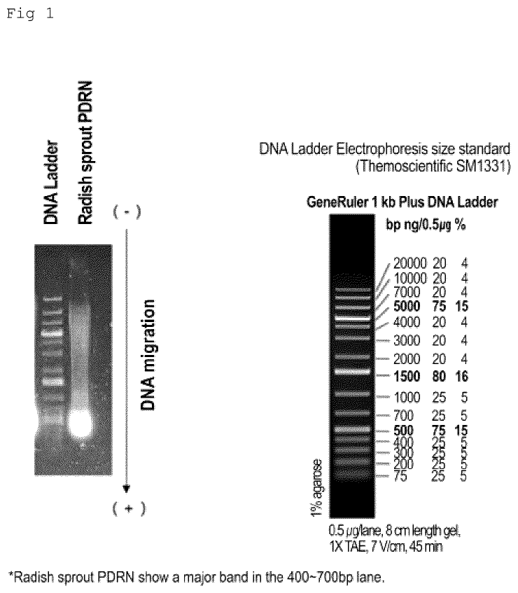

The extracted radish sprout-derived PDRN was quantified using Nanodrop, and the presence or absence of DNA was confirmed by electrophoresis of 10 μl of DNA on 0.5% agarose gel. As a result, it was confirmed that PDRN was in good condition and had an average size of 400 to 700 bp and a molecular weight of 250 to 650 kDa, as shown in FIG. 1.

Thereafter, Metagenome Amplicon Sequencing was performed using the obtained radish sprout PDRN by requesting Macrogen Co., Ltd. This test method is a cluster genome analysis method that can determine which species a specific DNA sample is composed of. It is a method that amplifies a specific region of a marker gene to create an amplicon library, and then analyzes the distribution and diversity through sequencing.

Library construction for sequencing was performed by amplifying rbcl2-rbcLa and ITS2F-ITS4R according to the illumine 16s metagenomics sequencing library protocol. As a result of the analysis, the DNA sample was analyzed as Raphanus sativus. A buffer solution for PCR amplification was added to 5 ng (rbcL2-rbcLa) and 10 ng (ITS2F-ITS4R) of gDNA, and 1 mM dNTP mix and 500 nM F/R PCR primers were added. The primer sequence information is as follows.

TABLE 1

| Prime | Sequence | |
|---|---|---|
| rbcL2 | tcgtcggcag cgtcagatgt gtataagaga cagcctacgg gnggcwgcag | 50 |
| | (SEQ ID NO: 3) | |
| rbcLa | gtctcgtggg ctcggagatg tgtataagag acaggactac hvgggtatct aatcc | 55 |
| | (SEQ ID NO: 4) | |
| ITS2F | tcgtcggcag cgtcagatgt gtataagaga cagatgcgat acttggtgtg aat | 53 |
| | (SEQ ID NO: 5) | |
| ITS4R | gtctcgtggg ctcggagatg tgtataagag acagtcctcc gcttattgat atgc | 54 |
| | (SEQ ID NO: 6) | |

As a result of metagenome amplicon sequencing, the radish sprout PDRN obtained in the present disclosure was analyzed to belong to the genus Raphanus at 99.9919% at the species level, and was confirmed to have the following representative genes.

TABLE 2

| SEQ ID NO | Sequence | |
|---|---|---|
| SEQ ID NO: | ctcaacccgg agttccacct gaagaagcag gggctgcggt agctgctgaa tcttctactg | 60 |
| 1 | gtacatggac aactgtgtgg accgatgggc ttaccagcct tgaccgttac aaaggccgat | 120 |
| | gctaccacat cgagcccgtt ccaggagaag aaactcaatt tattgcgtat gtagcttacc | 180 |
| | ccttagacct ttttgaagaa gggtctgtta ctaacatgtt tacctcaatt gtgggtaacg | 240 |
| | tatttgggtt caaagccctg gctgctctac gtctagagga tctgcgaatc cctccggctt | 300 |
| | atactaaaac tttccaggga ccacctcatg gtatccaagt tgaaagagat aaattgaaca | 360 |
| | agtatggacg tcccctatta ggatgtacta ttaaacctaa gttggggtta tccgcgaaga | 420 |
| | actatggtag agcagtttat gaatgtcta | 449 |
| SEQ ID NO: | ctcaacccgg agttccacct gaagaagcag gggctgcggt agctgctgaa tcttctactg | 60 |
| 2 | gtacatggac aaaactgtgt ggaccgatgg gcttaccagc cttgatcgtt acaaaggacg | 120 |
| | atgctaccac atcgagcccg ttccaggaga agaaaatcaa tttattgctt atgtagctta | 180 |
| | ccctttagac ctttttgaag aaggttcggt tactaacatg tttacctcga ttgtgggtaa | 240 |
| | tgtatttggg ttcaaagccc tggctgctct acgtctagag gatctgcgaa tccctcctgc | 300 |
| | ttatactcaa actttccagg gaccacctca tggtatccaa gttgaaagag agaaattgaa | 360 |
| | caagtatgga cgtcccctat taggatgtac tattaaacct aaattggggt tatccgcgaa | 420 |
| | gaactgtggt agagcagttt atgaatgtct a | 451 |

Plants of the genus Raphanus may include Raphanus sativus and Raphanus raphanistrum, and the Raphanus sativus is divided into numerous horticultural types, varieties, subspecies, and variants and grown or cultivated according to the characteristics of the climate or terrain, and representative examples include Raphanus sativus var. longipinnatus, Raphanus sativus var. sativus, Raphanus sativus var. caudatus, Raphanus sativus var. oleiformis, Raphanus sativus var. raphanistroides, and the like.

### Example 3. Confirmation of Cytotoxicity of Radish Sprout PDRN

The radish sprout PDRN was treated at different concentrations to perform cytotoxicity tests on human keratinocytes (HaCaT cells), A431 (keratinocyte-like; human epidermoid carcinoma), and fibroblasts (human dermal fibroblast, CCD-1064Sk; HDF). Each cell culture was performed according to conventional methods, basically in an incubator at 37° C. and 5% CO₂ incubator.

The experimental method for confirming cytotoxicity is as follows. Each cell was inoculated in a 24-well plate and cultured for 24 hours. The cultured cell medium was replaced with serum-free medium, and PDRN samples were treated at different concentrations and cultured for 24 hours. After the culture was completed, the cells were replaced with a medium containing a 10-fold dilution of a 2.5 mg/ml MTT solution and reacted. The supernatant was removed, 1 ml of DMSO was added to dissolve the produced MTT-formazan crystals, and the absorbance was measured at 570 nm using an ELISA reader. The control group used at this time used dimethyl sulfoxide (DMSO) as a dissolution solvent.

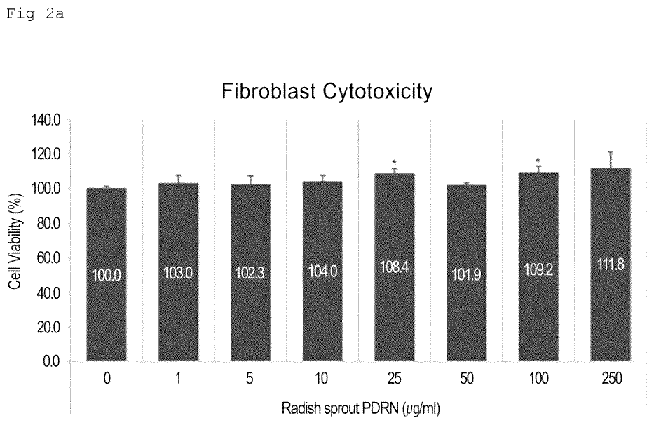

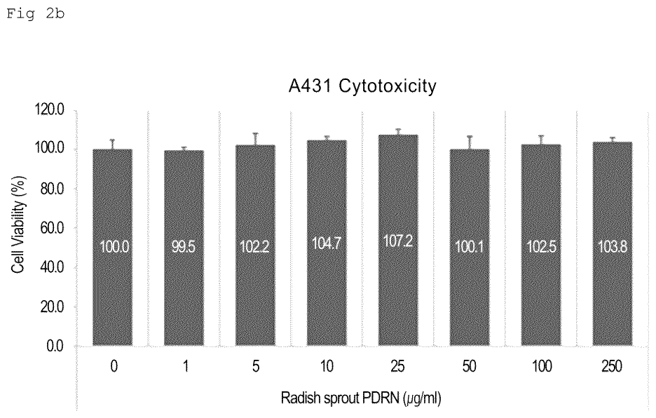

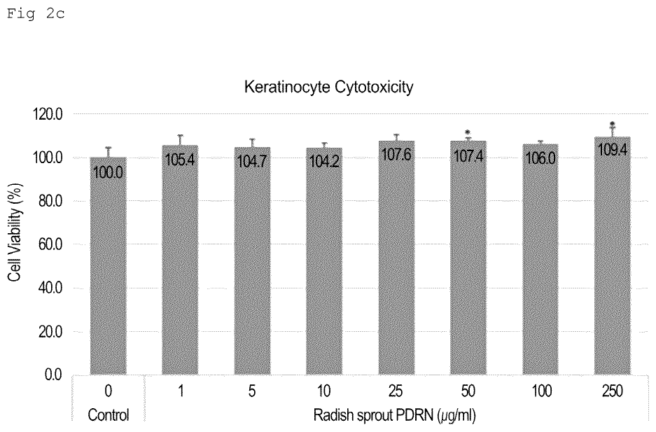

Cytotoxicity was expressed as a percentage of absorbance compared to the control, and the results are shown in FIGS. 2A to 2C.

TABLE 3
Fibroblast

| | Concentration | Average | Standard | |
|---|---|---|---|---|
| Sample | (μg/ml) | (%) | deviation | p-value |
| Control | | 100.0 | 1.29 | 1.0000 |
| Radish sprout | 1 | 103.0 | 4.33 | 0.3146 |
| PDRN | 5 | 102.3 | 4.91 | 0.4778 |
| | 10 | 104.0 | 3.45 | 0.1472 |
| | 25 | 108.4 | 2.89 | 0.0100 |
| | 50 | 101.9 | 1.42 | 0.1549 |
| | 100 | 109.2 | 3.27 | 0.0104 |
| | 250 | 111.8 | 9.23 | 0.0935 |

TABLE 4
A431

| | Concentration | Average | Standard | |
|---|---|---|---|---|
| Sample | (μg/ml) | (%) | deviation | p-value |
| Control | | 100.0 | 4.73 | 1.0000 |
| Radish sprout | 1 | 99.5 | 1.41 | 0.8771 |
| PDRN | 5 | 102.2 | 5.78 | 0.6316 |
| | 10 | 104.7 | 1.83 | 0.1810 |
| | 25 | 107.2 | 2.88 | 0.0860 |
| | 50 | 100.1 | 6.27 | 0.9812 |
| | 100 | 102.5 | 4.36 | 0.5334 |
| | 250 | 103.8 | 2.15 | 0.2731 |

TABLE 5
Keratinocyte

| | Concentration | Average | Standard | |
|---|---|---|---|---|
| Sample | (μg/ml) | (%) | deviation | p-value |
| Control | | 100.0 | 4.17 | 1.0000 |
| Radish sprout | 1 | 105.4 | 4.43 | 0.2003 |
| PDRN | 5 | 104.7 | 3.43 | 0.2096 |
| | 10 | 104.2 | 2.21 | 0.2006 |
| | 25 | 107.6 | 2.74 | 0.0574 |
| | 50 | 107.4 | 1.21 | 0.0413 |
| | 100 | 106.0 | 1.13 | 0.0725 |
| | 250 | 109.4 | 4.10 | 0.0496 |

As a result, it was confirmed that the radish sprout PDRN extracted in the present disclosure had no cytotoxicity to human keratinocytes, A431, and fibroblasts, with a viability of 99.5% or more in all cell and sample treatment groups up to 250 μg/ml of radish sprout PDRN.

### Example 4. Cell Basement Membrane and Skin Barrier Strengthening Efficacy of Radish Sprout PDRN

### Example 4-1. Enhancement Efficacy of Laminin-5 Protein

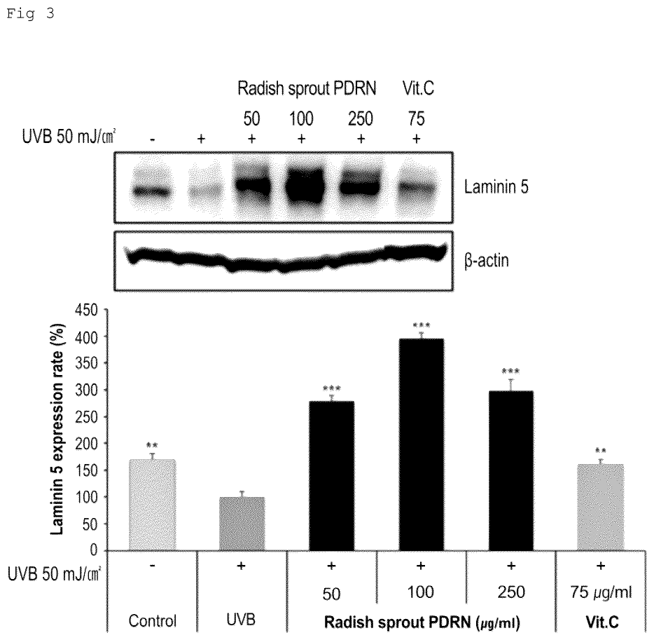

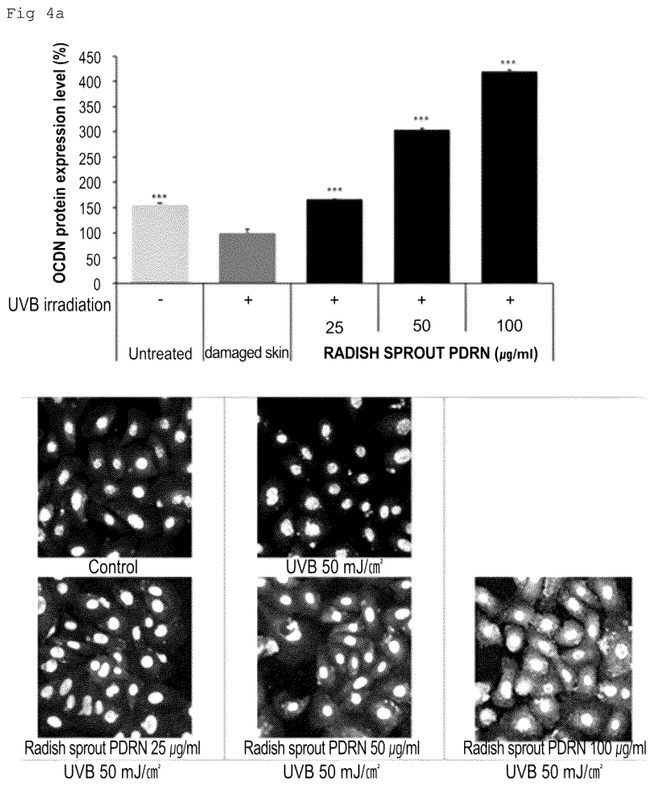

A431 cells were seeded on a 60 mm plate and cultured in MEM medium supplemented with 10% FBS at 37° C. in a 5% CO₂ incubator for 24 hours. After removing the medium from the plate, the plate was washed once with 1× Hanks' balanced salt solution (HBSS), and irradiated with UVB 50 mJ/cm² before treating with radish sprout PDRN. Thereafter, the medium was replaced with serum-free medium and treated with PDRN at different concentrations and cultured for 24 hours. After the culture was completed, the culture solution was removed, washed once with PBS (phosphate buffered saline) to remove the medium components, and then the cells were lysed using pro prep (iNtRON) buffer, proteins were extracted and quantified from the cells, and Western blotting was performed. Thereafter, the expression of laminin-5 protein, a factor related to skin barrier improvement, was confirmed. The results are shown in Table 6 and FIG. 3.

TABLE 6

| | Concentration | | Standard | |
|---|---|---|---|---|
| Sample | (μg/ml) | Average | deviation | p-value |
| Control | | 170.4 | 10.85 | 0.0012 |
| UVB 50 mJ/cm² | | 100.0 | 10.02 | 1.0000 |
| UVB + | 50 | 277.9 | 10.96 | 0.0003 |
| Radish sprout | 100 | 395.2 | 10.58 | 0.0000 |
| PDRN | 250 | 298.1 | 20.49 | 0.0001 |
| UVB + Vitamin C | 75 | 161.7 | 8.96 | 0.0060 |

The results of numerical substitution of the Western blot result bands showed that laminin-5 protein expression was significantly reduced in cells damaged by UVB treatment, but treatment with 50 to 250 μg/ml of radish sprout PDRN increased laminin-5 expression again by 2.7 to 4.0 times.

### Example 4-2. Enhancement Efficacy of Occludin (OCDN)

The protein expression level of occludin, a skin barrier factor, was directly confirmed within the cells. For this purpose, cells were cultured under sample treatment conditions similar to the laminin-5 experimental conditions. Primary normal human keratinocytes (NHEK cells) were seeded on 4-well slides, first irradiated with UVB 50 mJ/cm², and treated with PDRN at different concentrations, and the cells were cultured for 24 hours. Following this, the cells were fixed and permeabilized, and then immunostained sequentially with an anti-OCDN antibody and a secondary fluorescent antibody, and images were acquired using a fluorescence microscope.

TABLE 7

| | Concentration | | Standard | |
|---|---|---|---|---|
| Sample | (μg/ml) | Average | deviation | p-value |
| Control | | 157.2 | 1.06 | 0.0001 |
| UVB 50 mJ/cm² | | 100.0 | 6.81 | 1.0000 |
| (Damaged skin) | | | | |
| UVB + | 25 | 166.1 | 0.61 | 0.0001 |
| Radish sprout | 50 | 303.9 | 2.96 | 0.0000 |
| PDRN | 100 | 419.2 | 2.97 | 0.0000 |

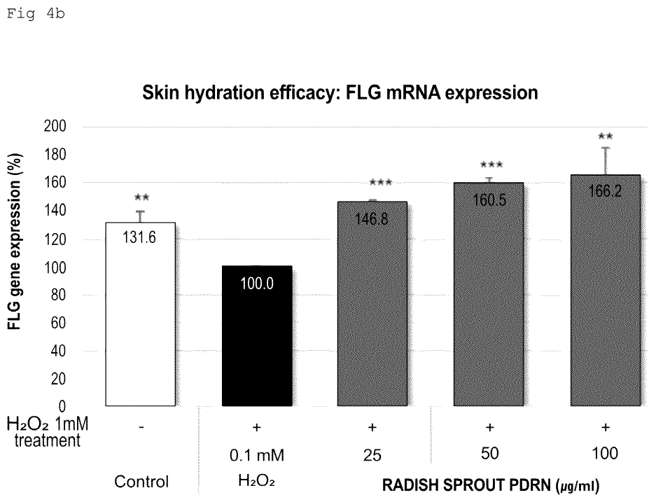

As a result, when examining the fluorescent staining photographs and the numerical results as shown in Table 7 and FIG. 4A, it can be confirmed that when treated with radish sprout PDRN, the protein expression level of occludin (OCDN) increases, and it is restored to the state before cell damage, and preferably increases by 1.6 to 4.2 times.

### Example 4-3. Enhancement Efficacy of Filaggrin (FLG)

Primary normal human keratinocytes (NHEK cells) were inoculated and cultured in a 6-well plate. The medium was replaced with serum-free medium, the samples were treated, and cultured for 24 hours at 37° C. in a 5% CO₂ incubator. After acutely oxidative stress (0.1 mM of H₂O₂) treatment, cells were collected with QIAzol™ lysis reagent (QIAGEN N.V.) and RNA was extracted according to the manufacturer's method. After quantifying the extracted RNA, cDNA was synthesized and real-time PCR (Applied Biosystems, 7500 FAST Real-Time PCR System) was performed to confirm gene expression (filaggrin).

TABLE 8

| | Concentration | |
|---|---|---|
| Sample | (μg/ml) | Average |
| Control | | 131.6 |
| H₂O₂ (0.1 mM) | | 100.0 |
| H₂O₂ + | 25 | 146.8 |
| Radish sprout | 50 | 160.5 |
| PDRN | 100 | 166.2 |

Table 8 and FIG. 4B show that 100 μg/ml of radish sprout PDRN increases filaggrin gene expression by 1.66-fold in damaged cells through H₂O₂ induction.

### Example 5. Antioxidant Efficacy of Radish Sprout PDRN-ROS Assay

Human keratinocytes (HaCaT cells) were inoculated and cultured in a 24-well plate. The medium was replaced with serum-free medium, the samples were treated, and cultured for 24 hours at 37° C. in a 5% CO₂ incubator. After treatment with H2DcFDA reagent and acutely oxidative stress (H₂O₂ 1 mM), the cells were washed with 1×HBSS and measured using an ELISA reader (Ex=485 nm, Em=535 nm).

TABLE 9

| | Concentration | | Standard | |
|---|---|---|---|---|
| Sample | (μg/ml) | Average | deviation | p-value |
| Control | | 74.8 | 2.38 | 0.0002 |
| H₂O₂ 1 mM | | 100.0 | 2.48 | 1.0000 |
| H₂O₂ + EGCG | 1 | 81.3 | 6.44 | 0.0093 |
| H₂O₂ + | 50 | 95.0 | 9.20 | 0.4120 |
| Radish sprout | 100 | 93.9 | 5.12 | 0.1367 |
| PDRN | 250 | 74.2 | 4.85 | 0.0012 |

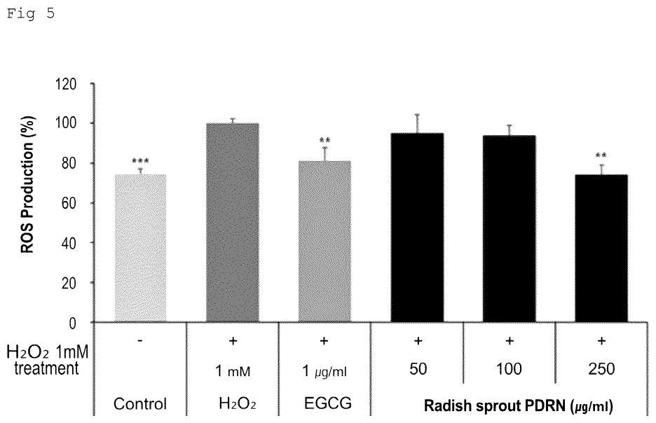

In Table 9 and FIG. 5, when treated with 250 μg/ml of radish sprout PDRN, the production of ROS in cells damaged by oxidative stress was reduced by 25.8%.

### Example 6. Detox Care of Radish Sprout PDRN—Confirmation of NRF-2Protein Expression

Nuclear factor erythroid 2-related factor 2 (NRF-2) is a transcription factor that regulates the expression of intracellular antioxidant and detoxification (Phase II) enzymes. Under normal conditions, it is degraded by the action of Keap1, but upon oxidative stimulation, it moves to the nucleus and its expression actually increases. Accordingly, the effect of radish sprout PDRN treatment on the expression of NRF-2 was confirmed.

To this end, human keratinocytes (HaCaT cells) were inoculated and cultured in a 60 mm dish, then replaced with serum-free medium, treated with H₂O₂ and the sample, and cultured at 37° C. in a 5% CO₂ incubator. The cells were scraped with a cell scraper, centrifuged, and the supernatant was discarded. Then, protein extraction solution (iNtRON) was added, centrifuged, and the supernatant was collected. The proteins were quantified, subjected to SDS-PAGE, and transferred to membranes. Then, the cells were blocked with 5% BSA or 5% skim milk and treated with primary antibodies [NRF-2, beta-actin]. Cells were washed with 1×TBST, treated with secondary antibodies, and finally washed with 1×TBST, treated with ECL solution, and then the bands were confirmed with Chemi-doc.

TABLE 10

| | Concentration | | Standard | |
|---|---|---|---|---|
| Sample | (μg/ml) | Average | deviation | p-value |
| Control | | 46.0 | 5.12 | 0.0001 |
| H₂O₂ (1 mM) | | 100.0 | 0.00 | 1.0000 |
| H₂O₂ + EGCG | 1.5 | 161.4 | 10.97 | 0.0006 |
| H₂O₂ + | 50 | 142.0 | 4.26 | 0.0001 |
| Radish sprout | 100 | 140.3 | 1.67 | 0.0000 |
| PDRN | 250 | 135.4 | 3.96 | 0.0001 |

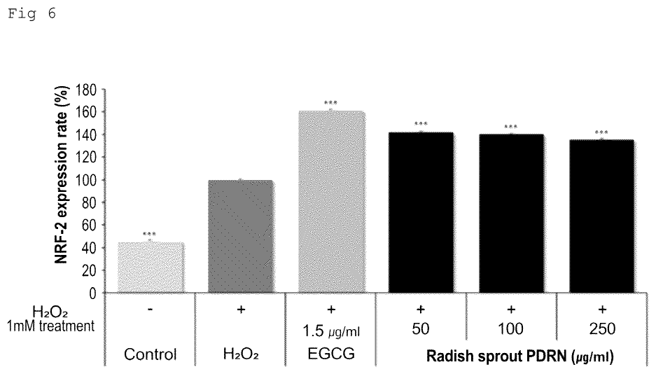

Table 10 and FIG. 6 show that oxidative stress induced by H₂O₂ activated the expression of NRF-2 protein, and that treatment with radish sprout PDRN further increased NRF-2 production.

The present disclosure relates to a cosmetic composition comprising PDRN extracted and purified from radish sprouts. The PDRN effectively removes ROS and further enhances the expression of NRF-2 induced by oxidative stress, thereby precisely regulating an intracellular antioxidant defense system. In addition, the radish sprout-derived PDRN exhibits excellent physiological activity that enhances the structural stability of the basement membrane and significantly improves skin barrier function. Accordingly, the radish sprout PDRN of the present disclosure can be easily applied to various cosmetic compositions requiring skin anti-oxidation, skin barrier protection and regeneration, and provides excellent efficacy in keeping the skin healthy from external stimuli.

What is claimed is:

1. An antioxidant cosmetic composition comprising polydeoxyribonucleotide (PDRN) extracted from radish sprouts.

2. The cosmetic composition of claim 1, wherein the PDRN has a reactive oxygen species scavenging ability.

3. The cosmetic composition of claim 1, wherein the PDRN further enhances nuclear factor erythroid 2-related factor 2 (NRF-2) activation induced by oxidative stress.

4. The cosmetic composition of claim 1, wherein the PDRN has a skin barrier strengthening efficacy.

5. The cosmetic composition of claim 1, wherein the PDRN enhances protein expression of laminin-5 or occludin or enhances gene expression of filaggrin.

6. A method for providing an antioxidant effect, comprising
applying to skin of a subject in need thereof the cosmetic composition according to claim 1.

7. The method according to claim 6, wherein the subject requires reactive oxygen species scavenging.

8. The method according to claim 6, wherein the subject requires enhancing nuclear factor erythroid 2-related factor 2 (NRF-2) activation induced by oxidative stress.

9. The method according to claim 6, wherein the subject requires skin barrier strengthening.

10. The method according to claim 6, wherein the subject requires enhancing protein expression of laminin-5 or occludin or enhances gene expression of filaggrin.
